(19) United States
(12) Patent Application Publication
(10) Pub. No.: US 2015/0313549 A1
(43) Pub. Date: Nov. 5, 2015

(54) HEART RATE MONITORING METHOD AND DEVCIE WITH MOTION NOISE SIGNAL REDUCTION

(71) Applicant: DIGIO2 INTERNATIONAL CO., LTD., Miaoli City (TW)

(72) Inventors: Cheng-Lung LEE, Yilan City (TW); Wen-Tse HUANG, Tainan City (TW); Jun-Min LO, Taipei City (TW)

(73) Assignee: DIGIO2 INTERNATIONAL CO., LTD., Miaoli City (TW)

(21) Appl. No.: 14/265,550

(22) Filed: Apr. 30, 2014

(51) Int. Cl. A61B 5/00; A61B 5/024

(52) U.S. Cl. CPC A61B 5/7214; A61B 5/02427; A61B 5/721; A61B 5/02438; A61B 5/681; A61B 2562/0219; A61B 2562/0238

(57) ABSTRACT

A heart rate monitoring method and device to analyze signals in time or frequency domain with motion noise signal reduction comprises at least two LEDs for providing two different light signals for incidenting into a portion of a human, a photodetector for detecting two reflected and scattered signals reflected and scattered form the human, and a processor for eliminating motion noise signal cause by any motion of the human. The processor may compare the two reflected and scattered signals and execute an independent component analysis to obtain a correct heart rate signal in time domain, and then the processor can calculate the correct heart rate. The processor may transform the two reflected and scattered signals form time domain to frequency domain and compare the two reflected and scattered signals in frequency domain to obtain a heart rate signal in frequency domain, and then the processor can calculate the heart rate.

### BACKGROUND OF THE INVENTION

1. Field of the Invention

The present invention relates to a heart rate monitoring method and device that is capable of eliminating motion noise signals of monitored heart rate signals.

2. Description of the Related Art

A heart rate of a human can be monitored according to an electrocardiogram (ECG) and a photoplethysmogram (PPG) in the clinical medicine. The heart rate of the human may be influenced by breath, blood pressure, motion, disease, or drugs taken. Therefore, a doctor can take care of a patient by monitoring his heart rate according to the ECG or the PPG. When using the ECG to monitor the heart rate of the human, electrodes are stuck at a surface of the human body to sense the heart rate. It is uncomfortable for the patient to have the electrodes put on his body.

Therefore, a non-invasive reflectance pulse oximetry is built for monitoring oxygen saturation of the patient. The reflectance pulse oximetry detects the oxygen saturation by incidenting a light signal output from a LED into the human body, and receiving a signal reflected and scattered from the human body. Then, the reflectance pulse oximetry can analyze the signal to obtain the oxygen saturation and a heart rate of the patient. The reflectance pulse oximetry may ray the light signal to different positions of the human body, such as a fingertip, an earlobe, a wrist, or a neck, for obtaining the oxygen saturation and the heart rate.

But, when the reflectance pulse oximetry is worn on the human body, such as the wrist, any motion of the human may cause a motion noise signal to influence the signal. Once the signal has been affected by the motion noise signal, the oxygen saturation and the heart rate may be incorrectly analyzed.

### SUMMARY OF THE INVENTION

An objective of the present invention is to provide a heart rate monitoring method and device with motion noise signal reduction for correctly detecting a heart rate of a human. The heart rate monitoring method and device can eliminate a motion noise signal to obtain a correct heart rate signal, and the heart rate can be calculated more precisely by analyzing the correct heart rate signal.

To achieve the foregoing objective, the heart rate monitoring method to analyze signals comprises the following steps:

providing a first light signal with a first wavelength and a second light signal with a second wavelength for incidenting into a portion of a human body;

detecting a first signal and a second signal reflected and scattered from the human body; wherein the first signal is a reflected and scattered signal of the first light signal, and the second signal is a reflected and scattered signal of the second light signal;

filtering the first signal and the second signal;

determining whether a motion noise signal is combined with the filtered first signal or the filtered second signal;

when the motion noise signal is not combined with the filtered first signal or the filtered second signal, calculating a heart rate according to the filtered first signal;

when the motion noise signal is combined with the filtered first signal or the filtered second signal, eliminating the motion noise signal from the filtered first signal and the filtered second signal to obtain a heart rate signal, and calculating the heart rate according to the heart rate signal.

The heart rate monitoring device for analyzing signals comprises at least one first LED, at least one second LED, a photodetector, and a processor electronically connected with the photodetector. Each first LED provides a first light signal with a first wavelength for incidenting into a portion of a human body, and each second LED provides a second light signal with a second wavelength for incidenting into the portion of the human body. The photodetector detects a first signal and a second signal reflected and scattered from the human body. The first signal is a reflected and scattered signal of the first light signal and the second signal is a reflected and scattered signal of the second light signal. In the embodiment, the photodetector may be a photodiode or a phototransistor.

The processor filters the first signal and the second signal, and determines whether a motion noise signal is combined with the filtered first signal or the filtered second signal.

When the motion noise signal is not combined with the filtered first signal or the filtered second signal, the processor calculates a heart rate according to the filtered first signal.

When the motion noise signal is combined with the filtered first signal or the filtered second signal, the processor eliminates the motion noise signal from the filtered first signal and the filtered second signal to obtain a heart rate signal, and calculates the heart rate according to the heart rate signal.

The heart rate monitoring device can be worn on a wrist of the human without using electrodes attached on the human body. Even if the human moves and the motion noise signal occurs, the present invention uses two different light signals to eliminate the motion noise signal. Therefore, the heart rate of the human can be correctly analyzed.

Other objectives, advantages and novel features of the invention will become more apparent from the following detailed description when taken in conjunction with the accompanying drawings.

### BRIEF DESCRIPTION OF THE DRAWINGS

FIGS. 1A, 1B and 1C are flowcharts of a heart rate monitoring method with motion noise signal reduction;

FIG. 2 is a schematic diagram of a human wearing the heart rate monitoring device with motion noise signal reduction;

FIGS. 3A and 3B are schematic side views of the heart rate monitoring device of FIG. 2;

FIG. 3C is a sectional schematic view of the heart rate monitoring device of FIG. 2;

FIG. 4 is a block diagram of a heart rate monitoring device of FIG. 2;

FIGS. 5A and 5B are waveforms of a first signal and a second signal reflected and scattered from a human body;

FIGS. 6A and 6B are waveforms of a filtered first signal and a filtered second signal;

FIG. 7 is a waveform of a reference signal;

FIG. 8A is a waveform of a motion noise signal;

FIG. 8B is a waveform of a heart rate signal;

FIG. 9A is a waveform of the filtered first signal of FIG. 6A after FFT;

FIG. 9B is a waveform of the filtered second signal of FIG. 6B after FFT;

FIG. 10 is a waveform of a heart rate signal in frequency domain.

### DETAILED DESCRIPTION OF THE INVENTION

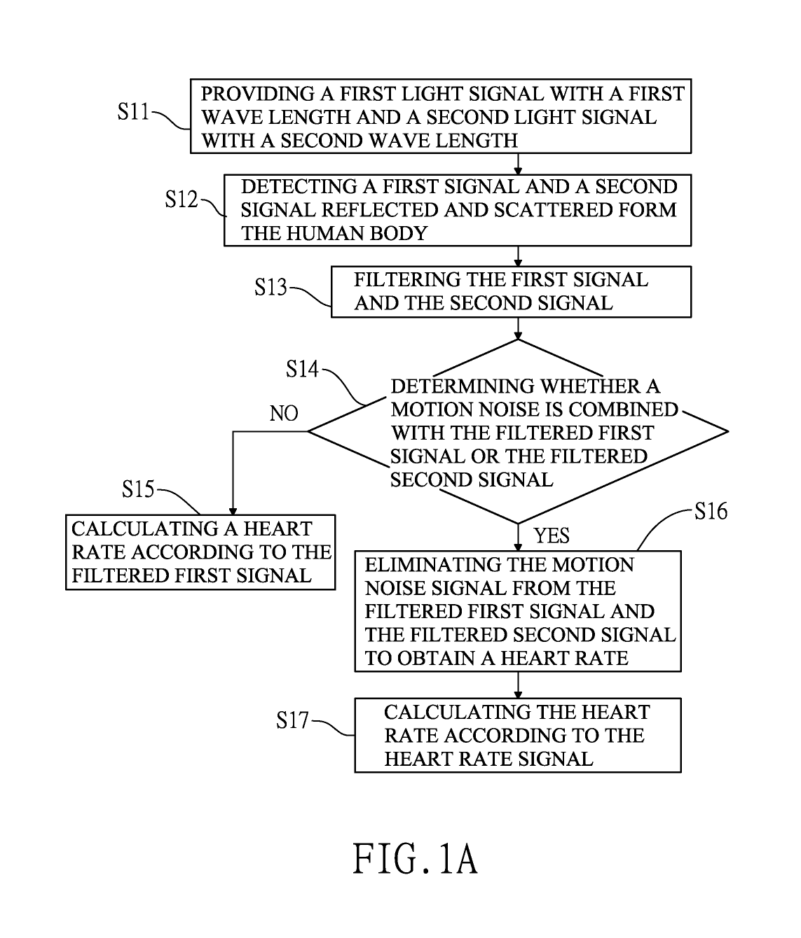

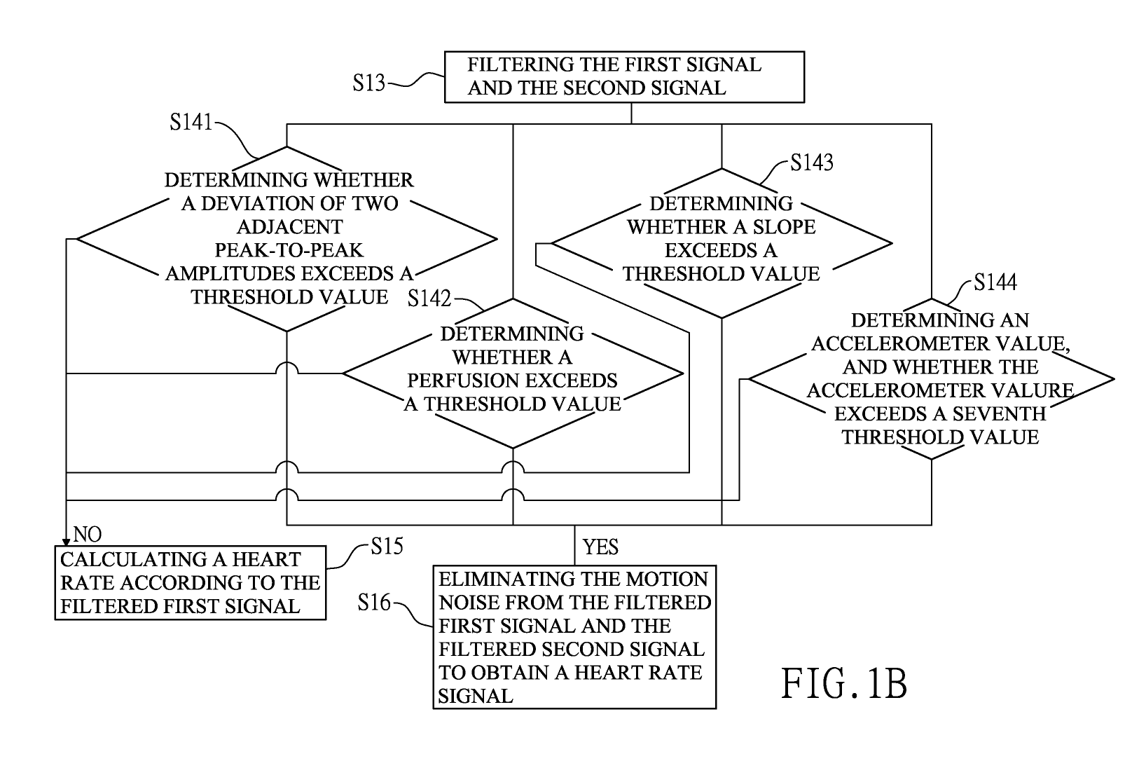

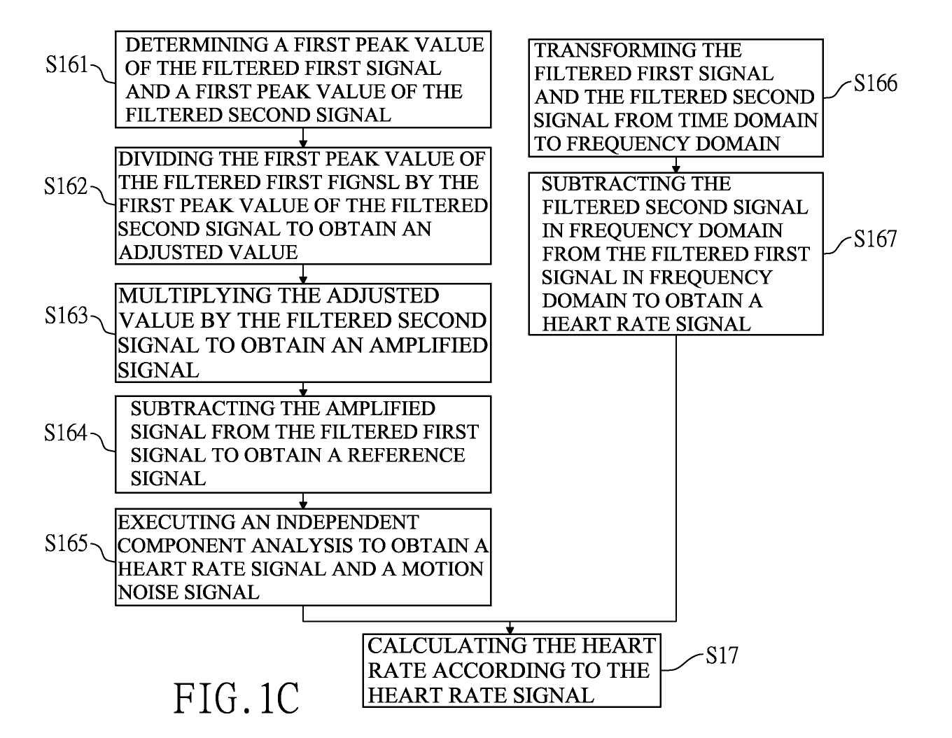

With reference to FIG. 1A, an embodiment of a heart rate monitoring method with motion noise signal reduction comprises the following steps:

providing a first light signal with a first wavelength and a second light signal with a second wavelength for incidenting into a portion of a human body (S11);

detecting a first signal and a second signal reflected and scattered from the human body (S12); wherein the first signal is a reflected and scattered signal of the first light signal, and the second signal is a reflected and scattered signal of the second light signal;

filtering the first signal and the second signal (S13);

determining whether a motion noise signal is combined with the filtered first signal or the filtered second signal (S14);

when the motion noise signal is not combined with the filtered first signal or the filtered second signal, calculating a heart rate according to the filtered first signal (S15); and

when the motion noise signal is combined with the filtered first signal or the filtered second signal, eliminating the motion noise signal from the filtered first signal and the filtered second signal to obtain a heart rate signal (S16), and calculating the heart rate according to the heart rate signal (S17).

With reference to FIG. 1B, the step of whether the motion noise signal is combined with the filtered first signal or the filtered second signal comprises the following steps:

determining whether a deviation of two adjacent peak-to-peak amplitudes of the filtered first signal exceeds a first threshold value (S141) or determining whether a deviation of two adjacent peak-to-peak amplitudes of the filtered second signal exceeds a second threshold value (S141);

when the deviation of two adjacent peak-to peak amplitudes of the filtered first signal is larger than the first threshold value or the deviation of two adjacent peak-to peak amplitudes of the filtered second signal is larger than the second threshold value, the motion noise signal is combined with the filtered first signal or the filtered second signal.

The step of whether the motion noise signal is combined with the filtered first signal or the second filtered signal comprises either the step S141 or the following steps:

determining whether a perfusion of the filtered first signal exceeds a third threshold value (S142) or whether a perfusion of the filtered second signal exceeds a fourth threshold value (S142); wherein the perfusion of the filtered first signal is calculated by dividing a peak-to-peak amplitude of the filtered first signal by a voltage value of a direct current of the filter first signal; and wherein the perfusion of the filtered second signal is calculated by dividing a peak-to-peak amplitude of the filtered second signal by a voltage value of a direct current of the filter second signal;

when the perfusion of the filtered first signal is larger than the third threshold value or the perfusion of the filtered second signal is larger than the fourth threshold value, the motion noise signal is combined with the filtered first signal or the filtered second signal;

The step of whether the motion noise signal is combined with the filtered first signal comprises any of the step S141, step S142 or the following steps:

determining whether a slope of a waveform of the filtered first signal exceeds a fifth threshold value (S143) or whether a slope of a waveform of the filtered second signal exceeds a sixth threshold value (S143);

when the slope of the waveform of the filtered first signal is larger than the fifth threshold value or the slope of the waveform of the filtered second signal is larger than the sixth threshold value, the motion noise signal is combined with the filtered first signal or the filtered second signal.

The step of whether the motion noise signal is combined with the filtered first signal comprises any of the step S141, step S142, step S143 or the following steps:

determining an accelerometer value, and determining whether the accelerometer value exceeds a seventh threshold value (S144);

when the accelerometer value is larger than the seventh threshold value, the motion noise signal is combined with the filtered first signal and the filtered second signal.

With reference to FIG. 1C, the motion noise signal can be eliminated either in the time domain or in the frequency domain. When the motion noise signal to be eliminated exists in time domain, the step of eliminating the motion noise signal from the filtered first signal and the filtered second signal to obtain the heart rate signal comprises the following steps:

determining a first peak value of the filtered first signal and a first peak value of the filtered second signal (S161);

dividing the first peak value of the filtered first signal by the first peak value of the filtered second signal to obtain an adjusted value (S162);

multiplying the adjusted value by the filtered second signal to obtain an amplified signal (S163);

subtracting the amplified signal from the filtered first signal to obtain a reference signal (S164);

executing an independent component analysis on the reference signal and the filtered first signal to obtain a heart rate signal and a motion noise signal (S165); and

calculating the heart rate according to the heart rate signal (S17).

When the motion noise signal to be eliminated exists in frequency domain, the step of eliminating the motion noise signal from the filtered first signal and the filtered second signal to obtain the heart rate signal comprises either the steps S161 to S165 or the following steps:

transforming the filtered first signal and the filtered second signal from the time domain to the frequency domain (S166);

subtracting the filtered second signal in the frequency domain from the filtered first signal in the frequency domain to obtain a heart rate signal in the frequency domain (S167); and

calculating the heart rate according to the heart rate signal in the frequency domain (S17). In the embodiment of the of the heart rate monitoring method with motion noise signal reduction, the frequency bands of the filtered first signal and the filtered second signal are between 0.5 Hz and 15 Hz, and the filtered first signal and the filtered second signal are transformed from the time domain to the frequency domain by the fast Fourier transform.

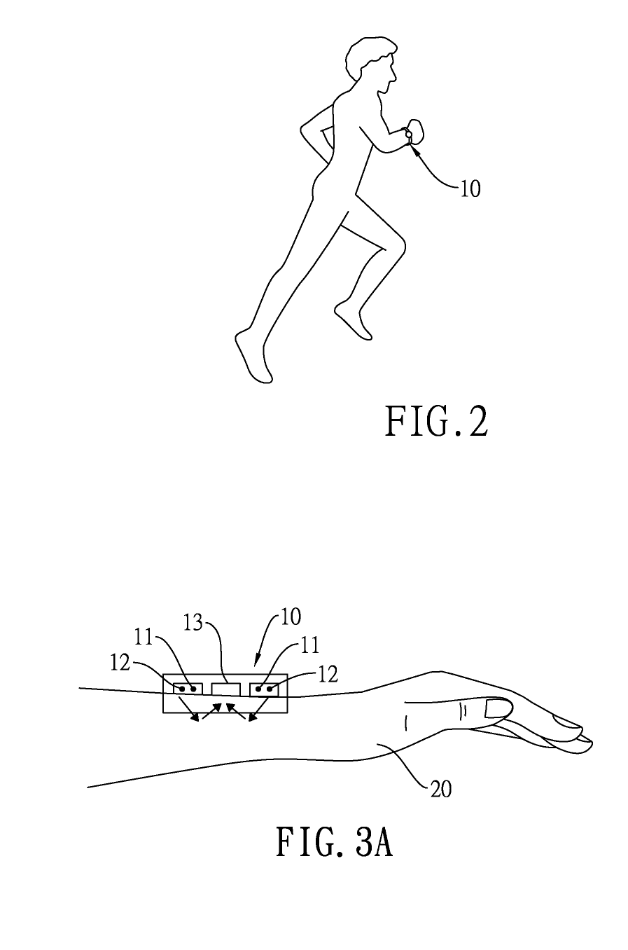

With reference to FIG. 2, an embodiment of a heart rate monitoring device 10 can execute the heart rate monitoring method, and can be wore like a watch on a wrist of a human 20.

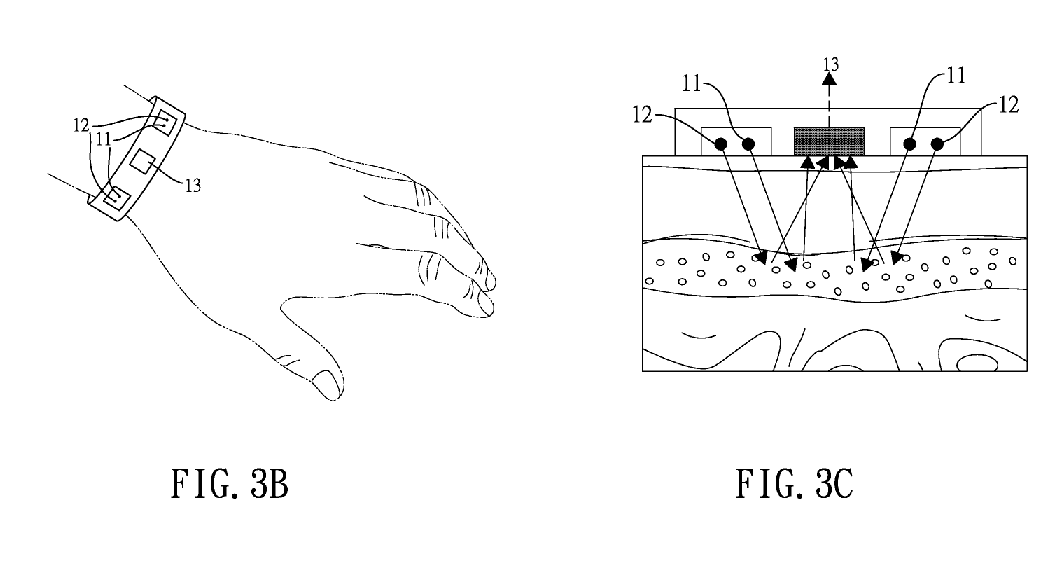

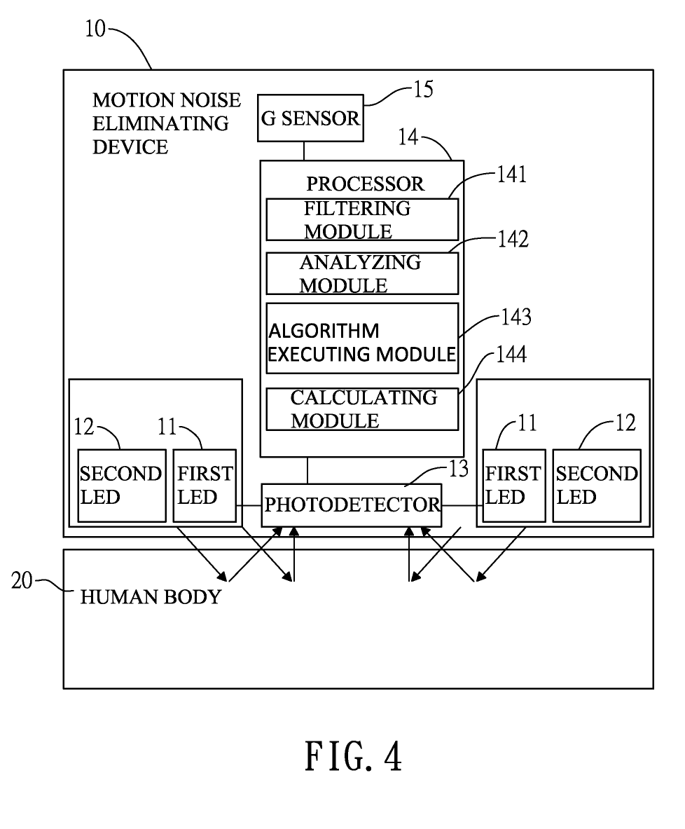

With reference to FIG. 3A, 3B, 3C and FIG. 4, the embodiment of the heart rate monitoring device 10 comprises at least one first LED 11 providing the first light signal for incidenting into a portion of the human body, at least one second LED 12 providing the second light signal for incidenting into the portion of the human body, a photodetector 13 for detecting the first signal and the second signal, and a processor 14 electronically connected with the photodetector 13. The first signal is a reflected and scattered signal of the first light signal reflected and scattered from the human body, and the second signal is a reflected and scattered signal of the second light signal reflected and scattered from the human body.

The processor 14 filters the first signal and the second signal, and determines whether the motion noise signal is combined with the filtered first signal or the filtered second signal. When the motion noise signal is not combined with the filtered first signal or the filtered second signal, the processor 14 calculates a heart rate according to the filtered first signal. When the motion noise signal is combined with the filtered first signal or the filtered second signal, the processor 14 eliminates the motion noise signal from the filtered first signal and the filtered second signal to obtain a heart rate signal, and calculates the heart rate according to the heart rate signal.

The processor 14 comprises a filtering module 141, an analyzing module 142, an algorithm executing module 143, and a calculating module 144. The filtering module 141 filters the first signal and the second signal. In the embodiment, the filtering module 141 filters the first signal and the second signal, and frequency bands of the filtered first signal and the filtered second signal are between 0.5 Hz and 15 Hz. The filtering module 141 may be a digital filter.

The analyzing module 142 determines whether a deviation of two adjacent peak-to-peak amplitudes of the filtered first signal exceeds a first threshold value or determines whether a deviation of two adjacent peak-to-peak amplitudes of the filtered second signal exceeds a second threshold value.

When the deviation of two adjacent peak-to peak amplitudes of the filtered first signal is lower than the first threshold value or the deviation of two adjacent peak-to peak amplitudes of the filtered second signal is lower than the second threshold value, the calculating module 144 calculates the heart rate according to the filtered first signal.

When the deviation of two adjacent peak-to peak amplitudes of the filtered first signal is larger than the first threshold value or the deviation of two adjacent peak-to peak amplitudes of the filtered second signal is larger than the second threshold value, the motion noise signal is combined with the filtered first signal or the filtered second signal, and the algorithm executing module 143 eliminating the motion noise signal from the filtered first signal and the filtered second signal to obtain the heart rate signal.

The analyzing module 142 may further determine whether a perfusion of the filtered first signal exceeds a third threshold value or whether a perfusion of the filtered second signal exceeds a fourth threshold value. The perfusion of the filtered first signal is calculated by dividing a peak-to-peak amplitude of the filtered first signal by a voltage value of a direct current of the filter first signal. The perfusion of the filtered second signal is calculated by dividing a peak-to-peak amplitude of the filtered second signal by a voltage value of a direct current of the filter second signal.

When the perfusion of the filtered first signal is lower than the third threshold value or the perfusion of the filtered second signal is lower than the fourth threshold value, the calculating module 144 calculates the heart rate according to the filtered first signal.

When the perfusion of the filtered first signal is larger than the third threshold value or the perfusion of the filtered second signal is larger than the fourth threshold value, the motion noise signal is combined with the filtered first signal or the filtered second signal, and the algorithm executing module 143 eliminating the motion noise signal from the filtered first signal and the filtered second signal to obtain the heart rate signal.

The analyzing module 142 may further determine whether a slope of a waveform of the filtered first signal exceeds a fifth threshold value or whether a slope of a waveform of the filtered second signal exceeds a sixth threshold value.

When the slope of the waveform of the filtered first signal is lower than the fifth threshold value or the slope of a waveform of the filtered second signal is lower than the sixth threshold value, the calculating module 144 calculates the heart rate according to the filtered first signal.

When the slope of the waveform of the filtered first signal is larger than the fifth threshold value or the slope of the waveform of the filtered second signal is larger than the sixth threshold value, the motion noise signal is combined with the filtered first signal or the filtered second signal, and the algorithm executing module 143 eliminating the motion noise signal from the filtered first signal and the filtered second signal to obtain the heart rate signal.

The heart rate monitoring device 10 further comprises a G sensor 15. The G sensor 15 is electronically connected with the processor 14, can detect any motion of the heart rate monitoring device 10, and outputs an accelerometer value. The analyzing module 142 may further determine the accelerometer value, and determine whether the accelerometer value exceeds a seventh threshold value.

When the accelerometer value is lower than the seventh threshold value, the calculating module 144 calculates the heart rate according to the filtered first signal.

When the accelerometer value is larger than the seventh threshold value, the motion noise signal is combined with the filtered first signal and the filtered second signal, the motion noise signal is combined with the filtered first signal and the filtered second signal, and the algorithm executing module 143 eliminating the motion noise signal from the filtered first signal and the filtered second signal to obtain the heart rate signal.

When the motion noise signal to be eliminated exists in the time domain, the algorithm executing module 143 determines a first peak value of the filtered first signal and a first peak value of the filtered second signal, divides the first peak value of the filtered first signal by the first peak value of the filtered second signal to obtain an adjusted value, multiplies the adjusted value by the filtered second signal to obtain an amplified signal, subtracts the amplified signal from the filtered first signal to obtain a reference signal, and executes an independent component analysis with inputs of the reference signal and the filtered first signal to obtain a heart rate signal and a motion noise signal. Then, the calculating module 144 calculates the heart rate according to the heart rate signal.

When the motion noise signal to be eliminated exists in the frequency domain, the algorithm executing module 143 transforms the filtered first signal and the filtered second signal from time domain to frequency domain, subtracts the filtered second signal in frequency domain from the filtered first signal in frequency domain to obtain a heart rate signal in frequency domain. Then, the calculating module 144 calculates the heart rate according to the heart rate signal in frequency domain. In the embodiment, the analyzing module 142 transforms the filtered first signal and the filtered second signal by the fast Fourier transform.

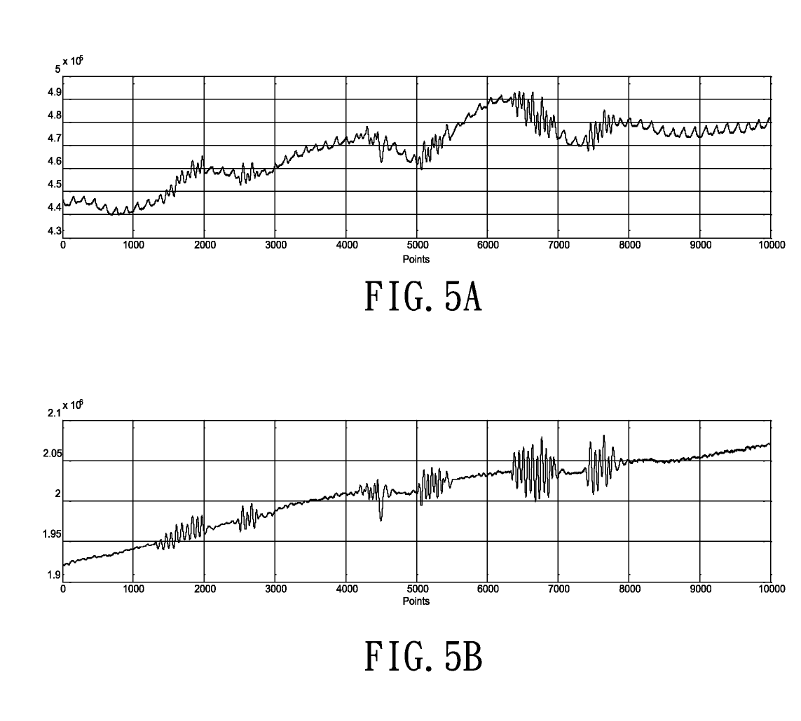

With reference to FIGS. 5A and 5B, the first LED 11 provides a green light, and the second LED 12 provides an orange light. A waveform of the first signal detected by the photodetector 13 is shown in FIG. 5A, and a waveform of the second signal detected by the photodetector 13 is shown in FIG. 5B. The green light is utilized for detecting the heart rate and the motion noise signal. Therefore, the first signal may combine the heart rate signal with motion noise signal. The orange light can detect the heart rate and the motion noise signal, and intensity of the motion noise signal detected by the orange light is stronger than intensity of the heart rate signal detected by the orange light. Therefore, the second signal may combine the heart rate signal with motion noise signal, but differs from the first signal.

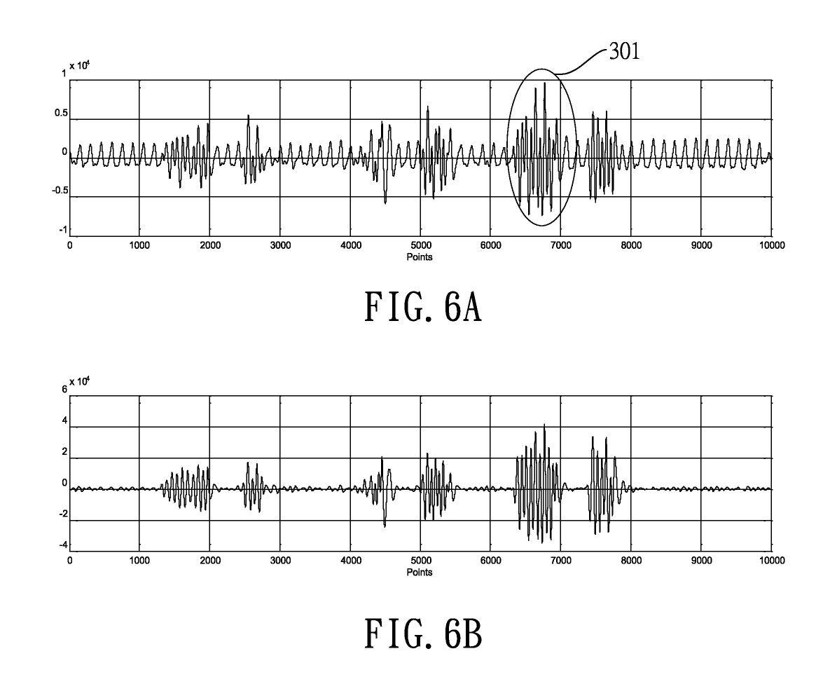

With reference to FIGS. 6A and 6B, when the filtering module 141 of the processor 14 filters the first signal and the second signal, the frequency band of the first signal and the second signal are between 0.5 Hz and 15 Hz. The filtered first signal is shown in FIG. 6A, and the second signal is shown in FIG. 6B. Then, the analyzing module 142 calculates the first amplitude and the second amplitude for determining whether the first amplitude exceeds the threshold value. When the first amplitude does not exceed the threshold, the motion noise signal combined in filtered first signal can be ignored, and the calculating module 144 can calculate the heart rate according to the filtered first signal. When the first amplitude exceeds the threshold, such as a first section 301 shown in FIG. 6A, the filtered first signal comprises the heart rate and the motion noise signal, and the motion noise signal is needed to be eliminated.

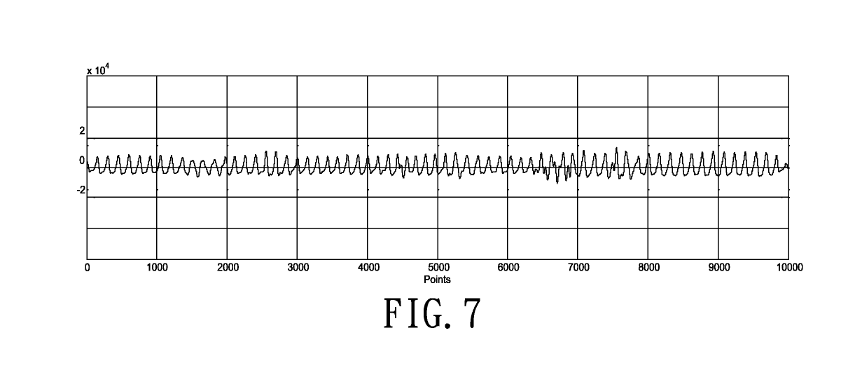

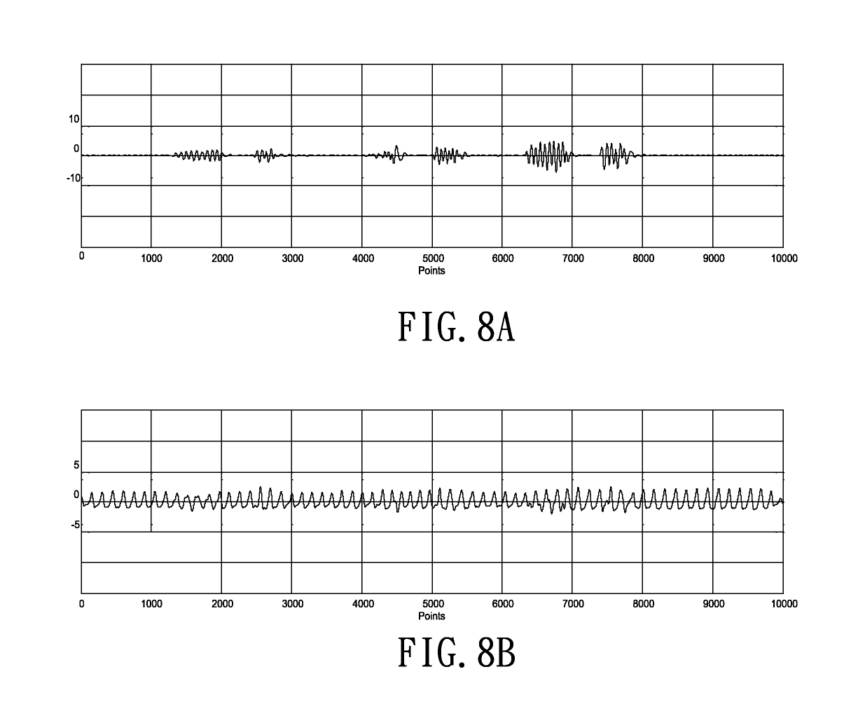

When the motion noise signal is eliminated in the time domain, a waveform of the reference signal is shown in FIG. 7, and the reference signal is calculated by the algorithm executing module 143. Then, the algorithm executing module 143 uses the reference signal and the filtered first signal as the inputs to execute the independent component analysis, and the results of the independent component analysis are shown in FIGS. 8A and 8B. FIG. 8A is the motion noise signal, and FIG. 8B is the heart rate signal. Therefore, the calculating module 144 can calculate the heart rate of the human 20 according to the heart rate signal.

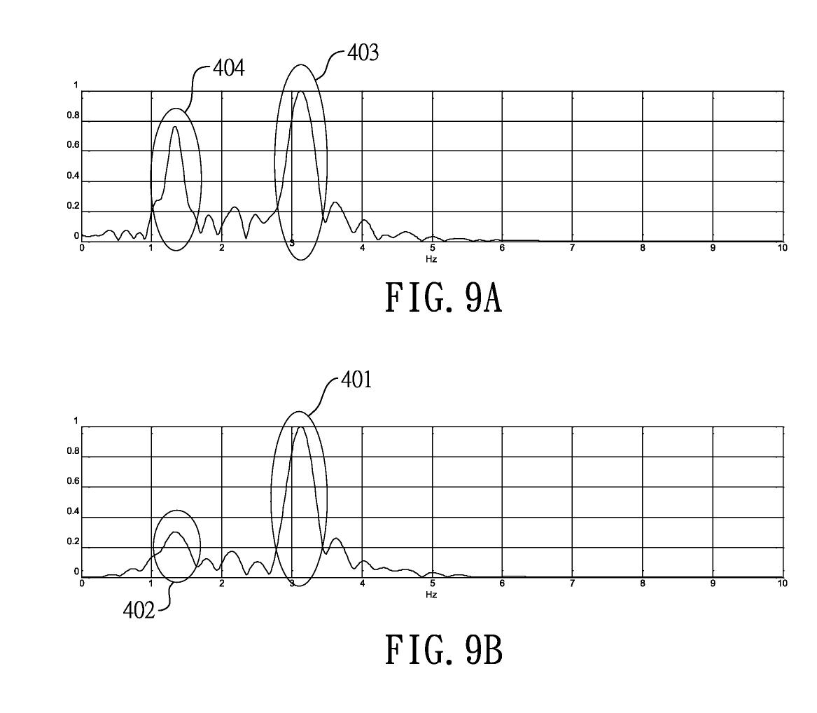

When the motion noise signal to be eliminated exists in the frequency domain, the filtered first signal is transformed by the fast Fourier transform, and a waveform of the filtered first signal in frequency domain is shown is FIG. 9A. The filtered second signal is transformed by the fast Fourier transform, and a waveform of the filtered second signal in frequency domain is shown is FIG. 9B. With reference to FIG. 9B, the intensity of the motion noise signal detected by the orange light is stronger than intensity of the heart rate signal detected by the orange light. With reference to FIGS. 9A and 9B, intensity of the heart rate signal detected by the green light is stronger than the intensity of the heart rate signal detected by the orange light. Therefore, a first frequency band 401 shown in FIG. 9B is the motion noise signal, and a second frequency band 402 shown in FIG. 9B is the heart rate signal. A third frequency band 403 of the filtered first signal in frequency domain shown in FIG. 9A corresponds to the first frequency band 401, and a fourth frequency band 404 of the filtered first signal in frequency domain shown in FIG. 9A corresponds to the second frequency band 402.

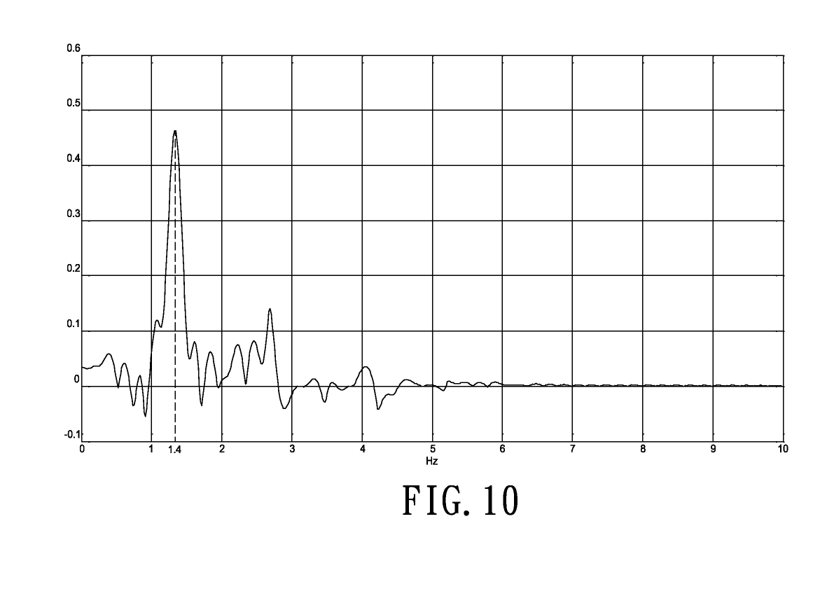

The algorithm executing module 143 subtracts the filtered second signal in frequency domain shown in FIG. 9B from the filtered first signal in frequency domain shown in FIG. 9A to obtain the heart rate signal shown in FIG. 10. Then, the calculating module 144 can multiply a frequency corresponding to a maximum intensity of the waveform shown in FIG. 10 by sixty to obtain a number of the heart rate (beat per minute; bpm). In FIG. 10, the frequency corresponds to the maximum intensity is 1.4 Hz, and the number of the heart rate is 84 bpm.

The present invention provides two different light signals, and detects the reflected and scattered signals for eliminating the motion noise signal. Therefore, when the human 20 wears the heart rate monitoring device 10 and moves his body causing the motion noise signal, the heart rate monitoring device 10 can eliminate the motion noise signal for obtaining a correct heart rate signal, and the heart rate of the human 20 can be correctly calculated according to the correct heart rate signal.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A heart rate monitoring method to analyze signals with motion noise signal reduction, comprising:
providing a first light signal with a first wavelength and a second light signal with a second wavelength for incidenting into a portion of a human body;
detecting a first signal and a second signal reflected and scattered from the human body; wherein the first signal is a reflected and scattered signal of the first light signal, and the second signal is a reflected and scattered signal of the second light signal;
filtering the first signal and the second signal;
determining whether a motion noise signal is combined with the filtered first signal or the filtered second signal;
when the motion noise signal is not combined with the filtered first signal or the filtered second signal, calculating a heart rate according to the filtered first signal;
when the motion noise signal is combined with the filtered first signal or the filtered second signal, eliminating the motion noise signal from the filtered first signal and the filtered second signal to obtain a heart rate signal, and calculating the heart rate according to the heart rate signal.

2. The heart rate monitoring method as claimed in claim 1, wherein the motion noise signal is eliminated in time domain, the step of eliminating the motion noise signal from the filtered first signal and the filtered second signal to obtain the heart rate signal comprises:
determining a first peak value of the filtered first signal and a first peak value of the filtered second signal;
dividing the first peak value of the filtered first signal by the first peak value of the filtered second signal to obtain an adjusted value;
multiplying the adjusted value by the filtered second signal to obtain an amplified signal;
subtracting the amplified signal from the filtered first signal to obtain a reference signal;
executing an independent component analysis on the reference signal and the filtered first signal to obtain the heart rate signal and a motion noise signal.

3. The heart rate monitoring method as claimed in claim 1, wherein the motion noise signal is eliminated in frequency domain, the step of eliminating the motion noise signal from the filtered first signal and the filtered second signal to obtain the heart rate signal comprises:
transforming the filtered first signal and the filtered second signal from time domain to frequency domain;
subtracting the filtered second signal in the frequency domain from the filtered first signal in the frequency domain to obtain the heart rate signal in the frequency domain.

4. The heart rate monitoring method as claimed in claim 1, wherein the step of determining whether the motion noise signal is combined with the filtered first signal or the filtered second signal comprises:
determining whether a deviation of two adjacent peak-to-peak amplitudes of the filtered first signal exceeds a first threshold value, wherein when the deviation of two adjacent peak-to peak amplitudes of the filtered first signal is larger than the first threshold value, the motion noise signal is combined with the filtered first signal.

5. The heart rate monitoring method as claimed in claim 1, wherein the step of determining whether the motion noise signal is combined with the filtered first signal or the filtered second signal comprises:
determining whether a deviation of two adjacent peak-to-peak amplitudes of the filtered second signal exceeds a second threshold value; wherein when the deviation of two adjacent peak-to-peak amplitudes of the filtered second signal is larger than the second threshold value, the motion noise signal is combined with the filtered second signal.

6. The heart rate monitoring method as claimed in claim 1, wherein the step of determining whether the motion noise signal is combined with the filtered first signal or the filtered second signal comprises:
determining whether a perfusion of the filtered first signal exceeds a third threshold value; wherein the perfusion is calculated by dividing a peak-to-peak amplitude of the filtered first signal by a voltage value of a direct current of the filter first signal; and wherein when the perfusion of the filtered first signal is larger than the third threshold value, the motion noise signal is combined with the filtered first signal.

7. The heart rate monitoring method as claimed in claim 1, wherein the step of determining whether the motion noise signal is combined with the filtered first signal or the filtered second signal comprises:
determining whether a perfusion of the filtered second signal exceeds a fourth threshold value; wherein the perfusion is calculated by dividing a peak-to-peak amplitude of the filtered second signal by a voltage value of a direct current of the filtered second signal; and wherein when the perfusion of the filtered second signal is larger than the fourth threshold value, the motion noise signal is combined with the filtered second signal.

8. The heart rate monitoring method as claimed in claim 1, wherein the step of determining whether the motion noise signal is combined with the filtered first signal or the filtered second signal comprises:
determining whether a slope of a waveform of the filtered first signal exceeds a fifth threshold value; wherein when the slope of the waveform of the filtered first signal is larger than the fifth threshold value, the motion noise signal is combined with the filtered first signal.

9. The heart rate monitoring method as claimed in claim 1, wherein the step of determining whether the motion noise signal is combined with the filtered first signal or the filtered second signal comprises:
determining whether a slope of a waveform of the filtered second signal exceeds a sixth threshold value; wherein when the slope of the waveform of the filtered second signal is larger than the sixth threshold value, the motion noise signal is combined with the filtered second signal.

10. The heart rate monitoring method as claimed in claim 1, wherein the step of determining whether the motion noise signal is combined with the filtered first signal or the filtered second signal comprises:
determining an accelerometer value, and determining whether the accelerometer value exceeds a seventh threshold value; wherein when the accelerometer value is larger than the seventh threshold value, the motion noise signal is combined with the filtered first signal and the filtered second signal.

11. The heart rate monitoring method as claimed in claim 2, wherein the step of determining whether the motion noise signal is combined with the filtered first signal or the filtered second signal comprises:
determining whether a deviation of two adjacent peak-to-peak amplitudes of the filtered first signal exceeds a first threshold value, wherein when the deviation of two adjacent peak-to peak amplitudes of the filtered first signal is larger than the first threshold value, the motion noise signal is combined with the filtered first signal.

12. The heart rate monitoring method as claimed in claim 2, wherein the step of determining whether the motion noise signal is combined with the filtered first signal or the filtered second signal comprises:
determining whether a deviation of two adjacent peak-to-peak amplitudes of the filtered second signal exceeds a second threshold value; wherein when the deviation of two adjacent peak-to-peak amplitudes of the filtered second signal is larger than the second threshold value, the motion noise signal is combined with the filtered second signal.

13. The heart rate monitoring method as claimed in claim 2, wherein the step of determining whether the motion noise signal is combined with the filtered first signal or the filtered second signal comprises:
determining whether a perfusion of the filtered first signal exceeds a third threshold value; wherein the perfusion is calculated by dividing a peak-to-peak amplitude of the filtered first signal by a voltage value of a direct current of the filter first signal; and wherein when the perfusion of the filtered first signal is larger than the third threshold value, the motion noise signal is combined with the filtered first signal.

14. The heart rate monitoring method as claimed in claim 2, wherein the step of determining whether the motion noise signal is combined with the filtered first signal or the filtered second signal comprises:
determining whether a perfusion of the filtered second signal exceeds a fourth threshold value; wherein the perfusion is calculated by dividing a peak-to-peak amplitude of the filtered second signal by a voltage value of a direct current of the filtered second signal; and wherein when the perfusion of the filtered second signal is larger than the fourth threshold value, the motion noise signal is combined with the filtered second signal.

15. The heart rate monitoring method as claimed in claim 2, wherein the step of determining whether the motion noise signal is combined with the filtered first signal or the filtered second signal comprises:
determining whether a slope of a waveform of the filtered first signal exceeds a fifth threshold value; wherein when the slope of the waveform of the filtered first signal is larger than the fifth threshold value, the motion noise signal is combined with the filtered first signal.

16. The heart rate monitoring method as claimed in claim 2, wherein the step of determining whether the motion noise signal is combined with the filtered first signal or the filtered second signal comprises:
determining whether a slope of a waveform of the filtered second signal exceeds a sixth threshold value; wherein when the slope of the waveform of the filtered second signal is larger than the sixth threshold value, the motion noise signal is combined with the filtered second signal.

17. The heart rate monitoring method as claimed in claim 2, wherein the step of determining whether the motion noise signal is combined with the filtered first signal or the filtered second signal comprises:
determining an accelerometer value, and determining whether the accelerometer value exceeds a seventh threshold value; wherein when the accelerometer value is larger than the seventh threshold value, the motion noise signal is combined with the filtered first signal and the filtered second signal.

18. The heart rate monitoring method as claimed in claim 3, wherein the step of determining whether the motion noise signal is combined with the filtered first signal or the filtered second signal comprises:
determining whether a deviation of two adjacent peak-to-peak amplitudes of the filtered first signal exceeds a first threshold value, wherein when the deviation of two adjacent peak-to peak amplitudes of the filtered first signal is larger than the first threshold value, the motion noise signal is combined with the filtered first signal.

19. The heart rate monitoring method as claimed in claim 3, wherein the step of determining whether the motion noise signal is combined with the filtered first signal or the filtered second signal comprises:
determining whether a deviation of two adjacent peak-to-peak amplitudes of the filtered second signal exceeds a second threshold value; wherein when the deviation of two adjacent peak-to-peak amplitudes of the filtered second signal is larger than the second threshold value, the motion noise signal is combined with the filtered second signal.

20. The heart rate monitoring method as claimed in claim 3, wherein the step of determining whether the motion noise signal is combined with the filtered first signal or the filtered second signal comprises:
determining whether a perfusion of the filtered first signal exceeds a third threshold value; wherein the perfusion is calculated by dividing a peak-to-peak amplitude of the filtered first signal by a voltage value of a direct current of the filter first signal; and wherein when the perfusion of the filtered first signal is larger than the third threshold value, the motion noise signal is combined with the filtered first signal.

21. The heart rate monitoring method as claimed in claim 3, wherein the step of determining whether the motion noise signal is combined with the filtered first signal or the filtered second signal comprises:
determining whether a perfusion of the filtered second signal exceeds a fourth threshold value; wherein the perfusion is calculated by dividing a peak-to-peak amplitude of the filtered second signal by a voltage value of a direct current of the filtered second signal; and wherein when the perfusion of the filtered second signal is larger than the fourth threshold value, the motion noise signal is combined with the filtered second signal.

22. The heart rate monitoring method as claimed in claim 3, wherein the step of determining whether the motion noise signal is combined with the filtered first signal or the filtered second signal comprises:
determining whether a slope of a waveform of the filtered first signal exceeds a fifth threshold value; wherein when the slope of the waveform of the filtered first signal is larger than the fifth threshold value, the motion noise signal is combined with the filtered first signal.

23. The heart rate monitoring method as claimed in claim 3, wherein the step of determining whether the motion noise signal is combined with the filtered first signal or the filtered second signal comprises:
determining whether a slope of a waveform of the filtered second signal exceeds a sixth threshold value; wherein when the slope of the waveform of the filtered second signal is larger than the sixth threshold value, the motion noise signal is combined with the filtered second signal.

24. The heart rate monitoring method as claimed in claim 3, wherein the step of determining whether the motion noise signal is combined with the filtered first signal or the filtered second signal comprises:
determining an accelerometer value, and determining whether the accelerometer value exceeds a seventh threshold value; wherein when the accelerometer value is larger than the seventh threshold value, the motion noise signal is combined with the filtered first signal and the filtered second signal.

25. The heart rate monitoring method as claimed in claim 1, wherein frequency bands of the filtered first signal and the filtered second signal are between 0.5 Hz and 15 Hz.

26. The heart rate monitoring method as claimed in claim 3, wherein the filtered first signal and the filtered second signal are transformed from the time domain to the frequency domain by the fast Fourier transform.

27. A heart rate monitoring device to analyze signals with motion noise signal reduction comprising:
at least one first LED; wherein each first LED provides a first light signal with a first wavelength for incidenting into a portion of a human;
at least one second LED; wherein each second LED provides a second light signal with a second wavelength for incidenting into the portion of the human;
a photodetector detecting a first signal and a second signal reflected and scattered from the human; wherein the first signal is a reflected and scattered signal of the first light signal, and the second signal is a reflected and scattered signal of the second light signal; and
a processor electronically connected with the photodetector, filtering the first signal and the second signal, and determining whether a motion noise signal is combined with the filtered first signal or the filtered second signal;
wherein when the motion noise signal is not combined with the filtered first signal or the filtered second signal, the processor calculates a heart rate according to the filtered first signal;
wherein when the motion noise signal is combined with the filtered first signal or the filtered second signal, the processor eliminates the motion noise signal from the filtered first signal and the filtered second signal to obtain a heart rate signal, and calculates the heart rate according to the heart rate signal.
